(19) United States
(12) Patent Application Publication
(10) Pub. No.: US 2025/0352399 A1
(43) Pub. Date: Nov. 20, 2025

(54) Sanitary Pads with a Perforated Top Sheet

(71) Applicant: Egal Pads Inc., Somerville, MA (US)

(72) Inventor: Thomas E. Devlin, Winchester, MA (US)

(21) Appl. No.: 19/206,439

(22) Filed: May 13, 2025

Related U.S. Application Data
(60) Provisional application No. 63/649,669, filed on May 20, 2024.

(51) Int. Cl. A61F 13/15; A61F 13/512

(52) U.S. Cl. CPC A61F 13/15211; A61F 13/5126; A61F 2013/15219; A61F 2013/15235

(57) ABSTRACT

Sanitary pads can be made flushable by means of perforation and stress concentrations in the layers to aid ripping before they a flushed into the sewer system, allowing absorbent materials to disperse instead expanding inside the pad and causing sewer blockage.

### CROSS REFERENCE TO RELATED APPLICATIONS

This application claims priority to U.S. Application No. 63/649,669, filed on May 20, 2024, the contents of which are hereby incorporated by reference in their entirety.

### FIELD OF THE INVENTION

The present invention relates to sanitary pads and their disposal.

### BACKGROUND

Legislation and public opinion has shifted with regards to sanitary pads, and it is becoming a more accepted practice that pads be made free and available next to all away-from-home toilets in the schools, workplaces, venues, and rest stops. There is a perception that this increase in availability of pads could increase the volume of waste, which is generally handled in bins located near or next to toilets. There is potential that blood borne pathogens could be hazardous to cleaners that empty those bins, and the presence of blood inside these bins is generally unsettling to users.

Thus, there here is a strong preference that used be pads be flushable, so that blood can be kept out of the trash stream. The problem is that pads are designed to soak up fluid, often much more than when in use, so that when they enter the sewer system, then can expand significantly and clog drains. Clogged drains are very costly for facilities.

One sanitary pad design uses materials that dissolve quickly into small fibers when immersed water so the pad cannot block the drain, and the small fibers can be processed in the sewer system. These have been proven to be ineffective in use because the weak materials tend to leak, causing blood to stain clothing.

So there is a need for a pad design that will not block sewers after flushing and can dissolve over time.

### SUMMARY OF THE INVENTION

A sanitary pad with a perforated top sheet that can be ripped apart before flushing will allow the internal absorbent materials to disperse and dissolve in the waste stream, instead of forming a large mass that can block drains. These absorbent materials generally comprise plant fibers that are compatible with sewage treatment facilities, such as wood pulp and cotton.

The top and bottom sheet materials would remain in the waste stream. But because they can no longer clog the pipes, they can take longer to dissolve, allowing them to be made of more durable materials that would not fail during use.

The top sheet of a sanitary pad is designed to be permeable, so there is no functional sacrifice to punch perforations that aid ripping and destruction of the pad before disposal. The bottom backing sheet cannot be perforated because it is designed to be impermeable.

In order to aid the start of the ripping, a non-perforated cut can be made in the ends or corners of the pad to create a starting tab. Because fluid spreads out from the center in a generally concentric pattern, the ends and corners will most likely be unsoiled, so the user can grasp the end and rip without getting blood on their fingers.

In another variation, the ends of each pad only require a slit to start ripping, and no other perforations. There is a stress concentration at the bottom of the slit which will aid the start of the rip. Because a rip may be hard to see, a notch can also serve as a stress concentration. This notch is a visual indicator that the pad is a flushable pad. Words and graphics can aid the message that the pad is flushable if it is ripped open.

### DESCRIPTION OF DRAWINGS

FIG. 1A is a representation of a typical sanitary pad showing the absorbing region, and the sealing region that surrounds it to keep fluid from leaking.

FIG. 1B is a variation of a typical sanitary pad showing wings that are used to wrap around an undergarment for a more secure placement.

FIG. 1C is a cross section of the sanitary pad of FIG. 1A showing the functional layers and regions. All cross-section thicknesses are scaled to be larger relative to the width for clarity of illustration.

FIG. 2A is a representation of a pad being dropped in a toilet and subsequent expansion after absorbing fluid in the sewer system.

FIG. 2B is representation of a ripped open pad being dropped in a toilet and that does not expand in the sewer system because its absorbing material is able to disperse.

FIG. 3A is depiction of the pad of FIG. 1A with two lines of perforations in the top permeable layer and a tab area designed for grasping and lifting in order to begin the ripping of the top permeable layer.

FIG. 3B is a representation of the pad of FIG. 3A being ripped open, exposing the absorbent material inside.

FIG. 4A is depiction of the pad of FIG. 1A with one line of perforation in the top permeable layer and a tab area in the corner designed for grasping and lifting in order to begin the ripping of the top permeable layer.

FIG. 4B is a representation of the pad of FIG. 4A being ripped open, exposing the absorbent material inside.

FIG. 5A is depiction of the pad of FIG. 1A with one line of perforation in the center of permeable layer and a line cut through all layers of the sealing region, providing a stress concentration that aids ripping of the entire pad.

FIG. 5B is depiction of the pad of FIG. 5A a notch punched through the sealing region that provides a stress concentration that aids ripping.

FIG. 5C is depiction of the pad of FIG. 1A a line cut through all layers of the sealing region, providing a stress concentration that aids ripping and printed graphics that instruct the user to rip before flushing.

FIG. 5D is a representation of the pads of FIG. 5A, FIG. 5B, and FIG. 5C being ripped open, exposing the absorbent material inside.

### DETAILED DESCRIPTION

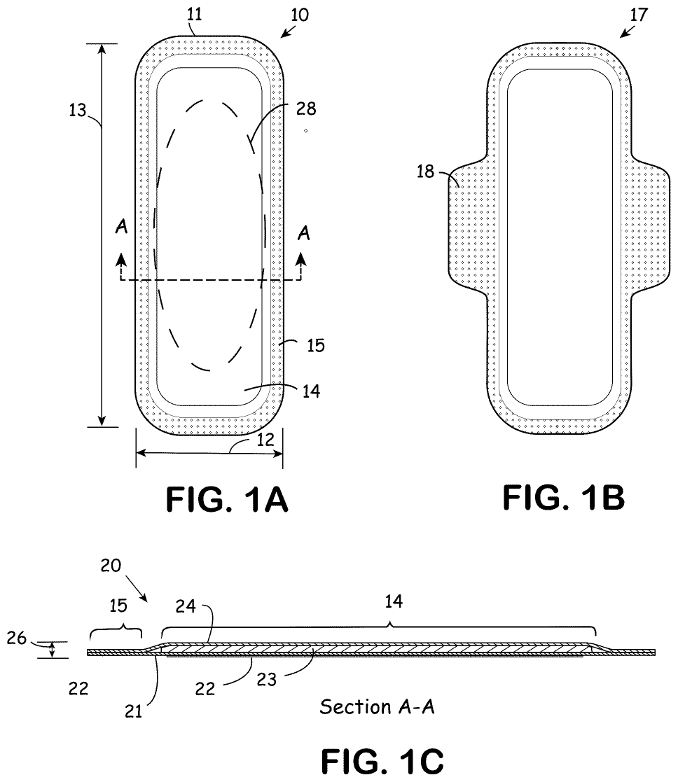

Referring to FIG. 1A, a menstrual pad 10 has a width 12 between 30 mm and 60 mm and a segment length 13 between 200 mm and 300 mm. The pad 10 includes absorbing region 14 having the purpose of absorbing and retaining fluids. The absorbing region 15 is surrounded by sealing region 15, the purpose of which is to retain the fluids in region 14. In some embodiments, the pad 10 has a die-cut shape 11.

Referring to FIG. 1B, an alternate die cut shape 11 for pad 10 has an extended wing region 18 that is configured to wrap around an undergarment for added retention.

Referring to Section A-A in FIG. 1A, the layers of a menstrual pad 10 include a backing layer 21 of fluid-impervious material with pressure sensitive adhesive 22 selectively attached to one face, and fluid absorbing material 23 attached to the opposite face in the absorbing region 14, and permeable material 24 attached to the opposite side of the fluid absorbing material 23, and which is welded to backing layer 21 in the sealing region 15 in order to create a seal that is impervious to fluid. The overall thickness 26 is generally thin, between 1 mm and 5 mm.

The adhesive is located where it is best suited for attaching the pad to an undergarment. Embodiments include those in which the fluid impervious material is a low density polyethylene film and the absorbent fabric is a non-woven cotton fabric. Among the embodiments are those in which the fluid absorbing material is cotton, those in which it is cellulose, and those in which it is a laminate or assembly comprising super absorbent polymer (SAP). SAP can absorb 300 times its weight and 30-60 times its volume of water. It can be sprayed on as a powder, sometimes called slush powder, in a continuous operation. This results in extremely thin pads that can absorb as much fluid as thicker pads that use cotton or cellulose.

In normal use, menstrual blood is deposited generally in the center of the pad. It spreads outwards 28 from the center, leaving the far ends, and especially the corners, free of blood, making them acceptable areas for handling the pad after use.

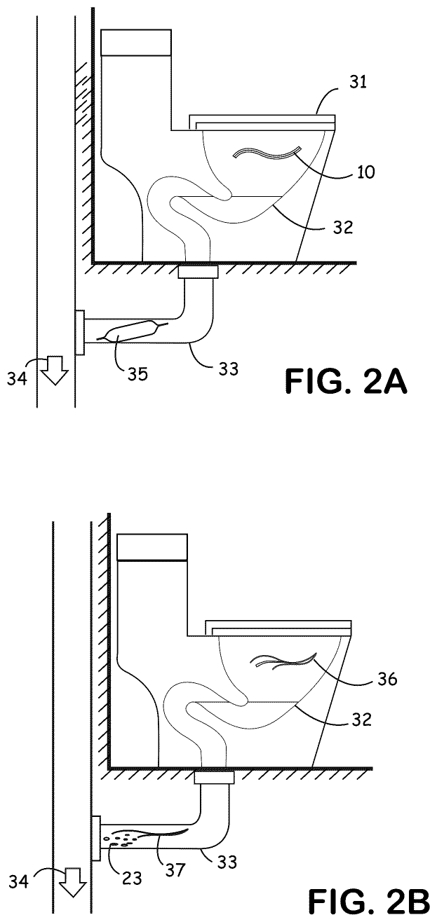

Referring to FIG. 2A, after use, and before flushing, a pad 10 has a generally small volume because it has absorbed a small amount of menstrual blood. After it is dropped in a toilet 31, it falls into water in the bottom of the bowl 32 and is subsequently flushed down sewer pipes 33 that connect to sewer-processing plants downstream 34. While immersed in water, the remaining absorbent material in the pads will reach its maximum capacity. As a result, the pad expands into a mass 35 that can block sewer lines.

Referring to FIG. 2B, after use, and before flushing, a pad 36 that has been ripped open will allow dispersion of absorbent material 23 inside the sewer lines 33 without clogging while the remaining layers 37 maintain a non-clogging configuration and can continue for further processing downstream 34.

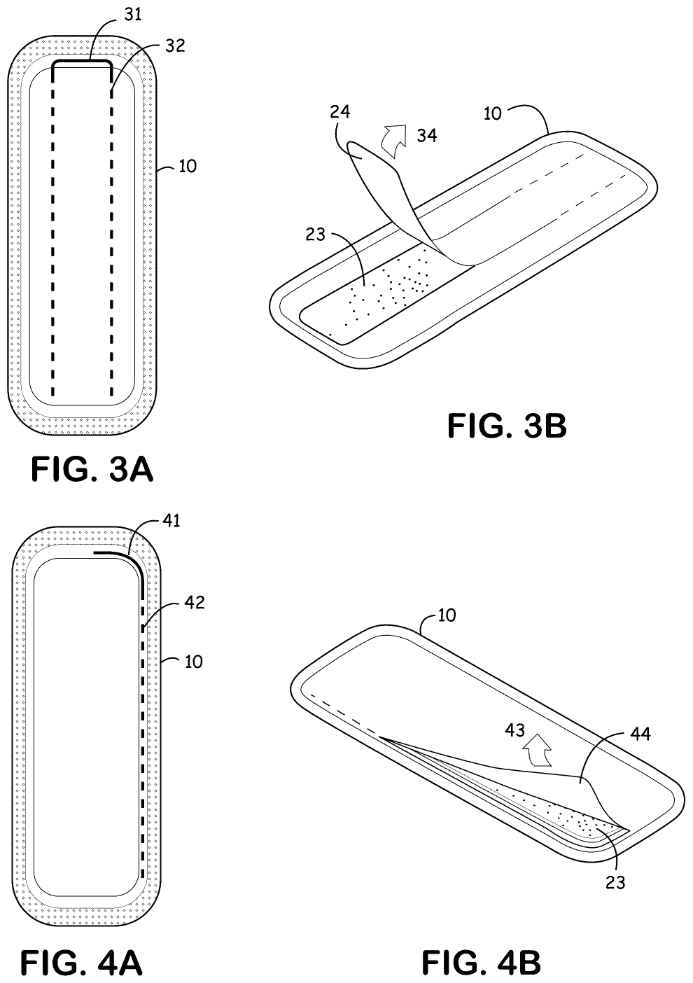

Referring to FIG. 3A, a pad 10 that is made ready to be ripped open will comprise a starter cut 31 through the top permeable layer only. This forms a tab that, when lifted, allows ripping to continue down centrally located perforated lines 32 through the top permeable layer only. In some embodiments, a second starter tab 31 is located on the opposite ends of lines 32 for symmetry.

Referring to FIG. 3B, once a flap 24 of the top permeable layer is removed by ripping 34, the inside absorbent material 23 is exposed to external forces and no longer confined to the pad assembly 10. This allows it to be dispersed in the sewer system.

Referring to FIG. 4A, a pad 10 that is made ready to be ripped open will comprise a starter cut in a corner 41 through the top permeable layer only that forms a tab that, when lifted, allows ripping to continue down a perforated line in the margin 42 through the top permeable layer only. Some embodiments feature a second starter tab 41 located on the opposite end of line 42 for symmetry.

Referring to FIG. 4B, once a flap 44 of the top permeable layer is removed by ripping 43, the inside absorbent material 23 is exposed to external forces and no longer confined to the pad assembly 10, allowing dispersion in the sewer system.

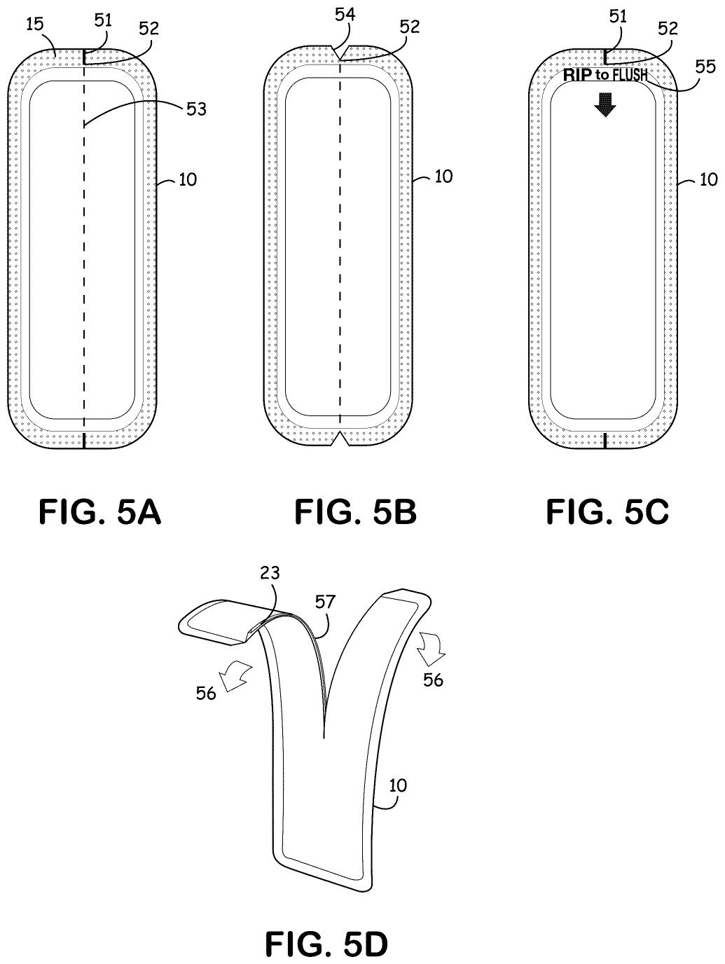

Referring to FIG. 5A, a pad 10 that is made ready to be ripped open will comprise a starter cut 51 through all layers of the sealed region 15 forming a stress concentration 52 that aids ripping and is aligned with a perforation 53 in the top permeable layer only. A second starter cut 51 can also be located on the opposite end of line 53 for symmetry.

Referring to FIG. 5B, the pad of FIG. 5A can alternatively use a notch 54 instead of a cut to form stress concentration 52 to aid ripping. The notch also serves a visual indicator of where to start ripping.

Referring to FIG. 5C, a pad 10 that is made to be ripped open can will comprise a starter cut 51 through all layers of the sealed region 15 forming a stress concentration 52 that aids ripping. Without perforation, ripping may be more difficult, but the forces required are not high. In order to remind users to rip the pad before flushing, graphics 55 can be printed on the pad.

Referring to FIG. 5D a pad 10 that has been pulled in opposing directions 56 will form a rip 57 that results in the absorbent material 23 being exposed to external forces and no longer confined to the pad assembly 10, allowing dispersion in the sewer system.

Depictions of ripping shown are not exact. Because of variations in properties of materials and the manner and direction of ripping, the final state of a ripped open pad can vary considerably from the depictions shown.

The details of an economical manufacturing method of these pads such as materials, bonding, adhesives, methods for attachment, perforating, and die-cutting have been left out for clarity, and it is assumed that those skilled in the art of pad manufacturing should be able to produce the pads described.

It is to be understood that the foregoing description is intended to illustrate and not to limit the scope of the invention, which is defined by the scope of the appended claims. Other embodiments are within the scope of the following claims.

1. A sanitary pad comprising an impermeable layer, an absorbent layer, a permeable layer, and a sealed region around the absorbent layer, wherein the sealed region is formed by bonding of the impermeable layer and the permeable layer, and where there is a weakening of at least one layer by a cut.

2. The sanitary pad of claim 1, wherein said weakening is formed by perforations in said permeable layer.

3. The sanitary pad of claim 1, wherein said weakening comprises a non-perforated cut is made in the permeable layer, wherein the non-perforated cut forms a tab that can be lifted.

4. The sanitary pad of claim 1 wherein said weakening is formed by a cut through the permeable and impermeable layer in the sealed region forming a stress concentration in the sealed region.

5. The sanitary pad of claim 1 wherein said weakening is formed by a die cut notch through the permeable and impermeable layer in the sealed region forming a stress concentration in the sealed region

6. A method comprising causing a sanitary pad to be destroyed after flushing the sanitary pad into a sewer system, wherein causing the sanitary pad to be destroyed comprises disrupting outer layers of the pad, thereby allowing internal absorbent materials within the pad to disperse in the sewer system after flushing.
